(19) United States
(12) Patent Application Publication
(10) Pub. No.: US 2023/0140043 A1
(43) Pub. Date: May 4, 2023

(54) ELECTRONIC DEVICE THAT PROVIDES CUSTOMIZED ERGONOMIC PROMPTS

(71) Applicant: MOTOROLA MOBILITY LLC, Wilmington, DE (US)

(72) Inventors: AMIT KUMAR AGRAWAL, BANGALORE (IN); JYOTHSNA BANDAMEEDIPALLI, BANGALORE (IN)

(21) Appl. No.: 17/514,546

(22) Filed: Oct. 29, 2021

(51) Int. Cl. G16H 20/30; G06K 9/00; G06T 7/11

(52) U.S. Cl. CPC G16H 20/30; G06K 9/00369; G06K 9/00375; G06K 9/00604; G06T 7/11; G06T 2207/30196; G06T 2207/30041

(57) ABSTRACT

An electronic device, computer program product, and method are provided that automatically visually monitors a person using the electronic device to provide health recommendations. The electronic device includes display device(s) and image capturing device(s) that produces an image stream and is configured to have a field of view that incorporates a person positioned to gaze at the display devices). A controller of the electronic device identifies at least one of a position or movement of at least one body part of the person incorporated in the image stream. The controller determines whether a health recommendation is associated with the position and/or movement of the at least one body part. The controller presents a prompt at the display device(s) requesting the person to reposition the at least one body part based on the health recommendation being associated with the position and/or movement of the at least one body part.

### BACKGROUND

### 1. Technical Field

The present disclosure relates generally to electronic devices, and more particularly to electronic devices that can visually recognize characteristics of and provide output directed to objects in an image stream produced by a local camera.

### 2. Description of the Related Art

Electronic devices such as mobile phones, network servers, desktop workstations, laptops, and tablets are powerful tools that are used for many functions, including work, entertainment, and communication. Some of these activities require extended periods of time positioned in front of a display device. In one example, electronic devices are often used to enable a user to participate in online human communication, which includes video-based communication where the electronic device is equipped with or connected to a local camera. So as to simulate an in-person conversation, meeting, or lecture, each participant in a video communication session generally positions themself within the field of view of the local camera and within detection range of the microphone. The respective image and audio streams are communicated to the video communication session for presentation by one or more second electronic devices. The participant needs to remain within proximity to the electronic device in a sedentary position while viewing the display device.

Recent trends are for increased use of video communications, including online meetings/sessions. Unlike attending in-person meetings, where the participants have to move from a home location or an office to another location to enable the face-to-face interaction, with online meetings, many people simply transition from one online work activity or communication session to another online work activity. The participant is able to make this transition without ever having to stand up or look away from the display device.

Excessive amount of time staring at a display screen can have detrimental health impacts on one's vision and overall health. With intent gazing, a person can even forget to blink as often as is recommended. The person can fail to periodically re-adjust eye focus to a more distant object, leading to eye strain and headaches. The person may tend to recline or stand with a slouching posture that is not ergonomically correct, leading to injury of the back and neck. The person may sit too long, increasing the risk of blood clots in the leg and other issues affecting health.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 depicts a functional block diagram of a communication environment including a communication device having components that automatically detects position or movement of at least one body portion of a person and responds with health recommendations, and within which the features of the present disclosure are advantageously implemented, according to one or more embodiments;

FIG. 2 depicts a functional block diagram of a network communication device that facilitates a video communication session and automatically manages healthy participation by a participant who is using a second communication device, according to one or more embodiments;

FIG. 3 depicts a communication environment of a host/teacher communication device, network communication device, and participant/student communication device during a video communication session, according to one or more embodiments;

FIG. 4 depicts the communication device visually identifying position and/or movement by a person and comparing the position/movement to library template images for posture and eye position, according to one or more embodiments;

FIG. 5 depicts the communication device having a user interface that presents prompts related to health recommendations for positioning and/or movement of the person who uses the communication device, according to one or more embodiments;

FIG. 6 depicts a flow diagram of a method performed by a host or network communication device for prompting healthy positioning and movement of a person using a second communication device during a video communication session, according to one or more embodiments;

FIG. 7A-7C (collectively FIG. 7) depicts a flow diagram of a method for prompting the person who uses the communication device, according to one or more embodiments;

FIG. 8 depicts a communication device providing background noise alerts to a person, according to one or more embodiments; and

FIG. 9 depicts a flow diagram of a method performed by a communication device for prompting avoidance of an excessively noisy environment, according to one or more embodiments.

### DETAILED DESCRIPTION

According to a first aspect of the present disclosure, an electronic device, a computer program product, and a method are provided that automatically visually monitors a person using the electronic device to provide health recommendations. The electronic device includes at least one image capturing device(s) that produces an image stream and is configured to have a field of view that incorporates a person positioned to use the electronic device (e.g., by gazing at an embedded or connected display devices). A controller of the electronic device identifies at least one of a position or movement of at least one body part of the person incorporated in the image stream. The controller determines whether a health recommendation is associated with the position and/or movement of the at least one body part. The controller presents one or more of a visual, tactile, or auditory prompt requesting the person to reposition the at least one body part based on the associated health recommendation.

In the following detailed description of exemplary embodiments of the disclosure, specific exemplary embodiments in which the various aspects of the disclosure may be practiced are described in sufficient detail to enable those skilled in the art to practice the invention, and it is to be understood that other embodiments may be utilized and that logical, architectural, programmatic, mechanical, electrical, and other changes may be made without departing from the spirit or scope of the present disclosure. The following detailed description is, therefore, not to be taken in a limiting sense, and the scope of the present disclosure is defined by the appended claims and equivalents thereof. Within the descriptions of the different views of the figures, similar elements are provided similar names and reference numerals as those of the previous figure(s). The specific numerals assigned to the elements are provided solely to aid in the description and are not meant to imply any limitations (structural or functional or otherwise) on the described embodiment. It will be appreciated that for simplicity and clarity of illustration, elements illustrated in the figures have not necessarily been drawn to scale. For example, the dimensions of some of the elements are exaggerated relative to other elements.

It is understood that the use of specific component, device and/or parameter names, such as those of the executing utility, logic, and/or firmware described herein, are for example only and not meant to imply any limitations on the described embodiments. The embodiments may thus be described with different nomenclature and/or terminology utilized to describe the components, devices, parameters, methods and/or functions herein, without limitation. References to any specific protocol or proprietary name in describing one or more elements, features or concepts of the embodiments are provided solely as examples of one implementation, and such references do not limit the extension of the claimed embodiments to embodiments in which different element, feature, protocol, or concept names are utilized. Thus, each term utilized herein is to be given its broadest interpretation given the context in which that term is utilized.

As further described below, implementation of the functional features of the disclosure described herein is provided within processing devices and/or structures and can involve use of a combination of hardware, firmware, as well as several software-level constructs (e.g., program code and/or program instructions and/or pseudo-code) that execute to provide a specific utility for the device or a specific functional logic. The presented figures illustrate both hardware components and software and/or logic components.

Those of ordinary skill in the art will appreciate that the hardware components and basic configurations depicted in the figures may vary. The illustrative components are not intended to be exhaustive, but rather are representative to highlight essential components that are utilized to implement aspects of the described embodiments. For example, other devices/components may be used in addition to or in place of the hardware and/or firmware depicted. The depicted example is not meant to imply architectural or other limitations with respect to the presently described embodiments and/or the general invention. The description of the illustrative embodiments can be read in conjunction with the accompanying figures. Embodiments incorporating teachings of the present disclosure are shown and described with respect to the figures presented herein.

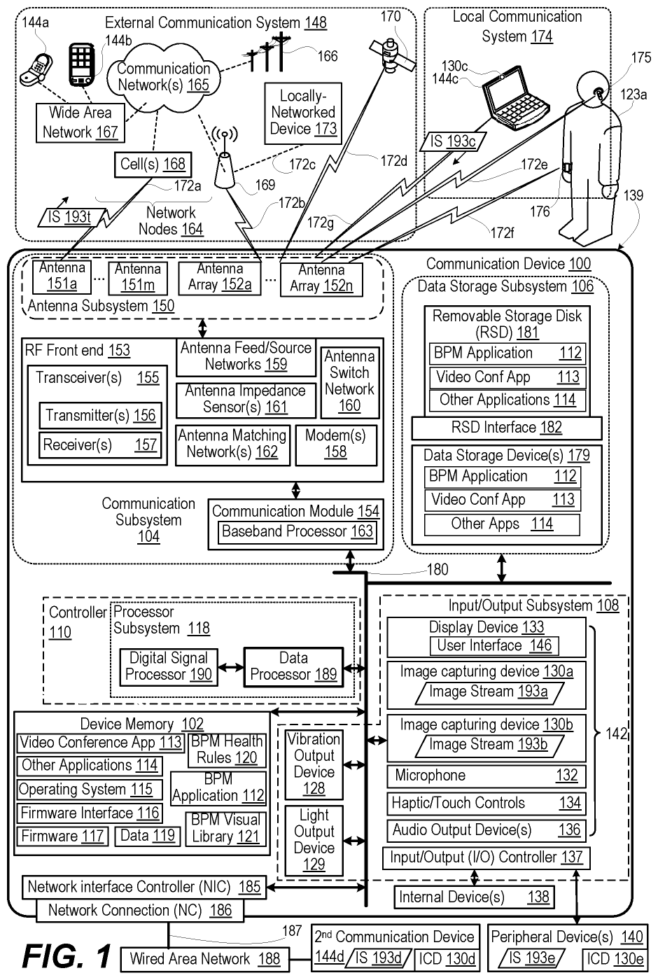

FIG. 1 depicts a functional block diagram of an electronic device, specifically communication device 100, within an operating environment and within which several of the features of the present disclosure are advantageously implemented. Referring now to the specific component makeup and the associated functionality of communication device 100. In one or more embodiments, communication device 100 has device memory 102, communication subsystem 104, data storage subsystem 106, and input/output (I/O) subsystem 108. Device memory 102 and each subsystem (104, 106, and 108) are managed by controller 110. Device memory 102 includes program code for applications, such as body position/movement (BPM) application 112, video conference application 113, and other application(s) 114. Device memory 102 further includes operating system (OS) 115, firmware interface 116, such as basic input/output system (BIOS) or Uniform Extensible Firmware Interface (UEFI), and firmware 117.

Controller 110 includes processor subsystem 118 that executes program code to provide operating functionality of communication device 100 that automatically detects positioning and movement by a person by visually identifying objects that include body part of the person in an image stream and generates alerts in response to positions/movements that are not in line with health recommendations. The software and/or firmware modules have varying functionality when their corresponding program code is executed by processor subsystem 118 or secondary processing devices within communication device 100. Processor subsystem 118 of controller 110 can execute program code of BPM application 112, video conference application 113, and other application(s) 114 to configure communication device 100 to perform specific functions. Device memory 102 can include data 119 used by the applications. In one or more embodiments, BPM application 112 can use BPM health rules data 120 and BPM visual library 121 to generate alerts for positioning and movement of person 123.

I/O subsystem 108 includes user interface components such as vibration output device 128, light output device 129, image capturing devices 130a-130b, microphone 132, display device 133, touch/haptic controls 134, and audio output device(s) 136. In an example, image capturing devices 130a-130b are front and back cameras. In another example, image capturing devices 130a-130b are on the same side but have different lenses such as two different ones of telephoto, wide angle, macro, or general purpose lenses. I/O subsystem 108 also includes I/O controller 137.

I/O controller 137 provides communication and power signals to functional components described herein as part of device memory 102, communication subsystem 104, data storage subsystem 106, or I/O subsystem 108. I/O controller 137 connects to internal devices 138, which are internal to housing 139, and to peripheral devices 140, such as external speakers, which are external to housing 139 of communication device 100. Internal devices 138 include computing, storage, communication, or sensing components depicted within housing 139. I/O controller 137 supports the necessary configuration of connectors, electrical power, communication protocols, and data buffering to act as an interface between internal devices 138 and peripheral devices 140 and other components of communication device 100 that use a different configuration for inputs and outputs.

Communication device 100 is managed by controller 110, which is communicatively coupled to image capturing devices 130a-130b and to at least one user interface device 142, such as at least one microphone 132. Image capturing device 130a and user interface device 142 allows a participant using communication device 100 to be an active participant in a video communication session with a second participant using a corresponding one of second communication device(s) 144a-144d that can be similarly configured and/or provide similar functionality as communication device 100. Controller 110 is also communicatively coupled to at least one display device 133 that presents user interface 146 for the video communication session. In an example, controller 110 executes BPM application 112 and video conference application 113 stored in device memory 102 to configure communication device 100 to enable health related alerts and communication respectively with second communication device(s) 144a-144d in the video communication session. It is appreciated that second communication device(s) 144a-144d can be identically or similarly equipped with components and functionality described for communication device 100. According to aspects of the present disclosure, communication device 100 can be communicatively coupled to one or more of image capturing devices (“ICD”) 130c-130e respectively provided for example by communication devices 144c/144d and peripheral device 140.

Each of communication device 100 and second communication device(s) 144a-144d can be one of a host of different types of devices, including but not limited to, a mobile cellular phone, satellite phone, or smart-phone, a laptop, a net-book, an ultra-book, a networked smart watch or networked sports/exercise watch, and/or a tablet computing device or similar device that can include wireless and/or wired communication functionality. As an electronic device supporting wireless communication, communication device 100 can be utilized as, and also be referred to as, a system, device, subscriber unit, subscriber station, mobile station (MS), mobile, mobile device, remote station, remote terminal, user terminal, terminal, user agent, user device, a Session Initiation Protocol (SIP) phone, a wireless local loop (WLL) station, a personal digital assistant (PDA), computer workstation, a handheld device having wireless connection capability, a computing device, or other processing devices connected to a wireless modem. Within the description of the remaining figures, references to similar components presented in a previous figure are provided the same reference numbers across the different figures. Where the named component is presented with different features or functionality, a different reference numeral or a subscripted reference numeral is provided (e.g., 100a in place of 100).

Referring now to the communication components and features of communication device 100. Communication subsystem 104 of communication device 100 enables wireless communication with external communication system 148. Communication sub system 104 includes antenna subsystem 150 having lower band antennas 151a-151m and higher band antenna arrays 152a-152n that can be attached in/at different portions of housing 139. Communication subsystem 104 includes radio frequency (RF) front end 153 and communication module 154. RF front end 153 includes transceiver(s) 155, which includes transmitter(s) 156 and receiver(s) 157. RF front end 153 further includes modem(s) 158. RF front end 153 includes antenna feed/source networks 159, antenna switch network 160, antenna impedance sensor(s) 161, and antenna matching network(s) 162. Communication module 154 of communication sub system 104 includes baseband processor 163 that communicates with controller 110 and RF front end 153. Baseband processor 163 operates in a baseband frequency range to encode data for transmission and decode received data, according to a communication protocol. Modem(s) 158 modulate baseband encoded data from communication module 154 onto a carrier signal to provide a transmit signal that is amplified by transmitter(s) 156. Modem(s) 158 demodulates each signal received from external communication system 148 using by antenna subsystem 150. The received signal is amplified and filtered by receiver(s) 157, which demodulate received encoded data from a received carrier signal. Antenna feed/source networks 159 transmits or receives from particular portions of antenna subsystem 150 and can adjust a phase between particular portions of antenna subsystem 150. Antenna switch network 160 can connect particular combinations of antennas (151a-151m, 152a-152n) to transceiver(s) 155. Controller 110 can monitor changes in antenna impedance detected by antenna impedance sensor(s) 161 for determining portions of antenna subsystem 150 that are blocked. Antenna matching network(s) 162 are connected to particular lower band antennas 151a-151m to tune impedance respectively of lower band antennas 151a-151m to match impedance of transceiver(s) 155. Antenna matching network(s) 162 can also be used to detune the impedance of lower band antennas 151a-151m to not match the impedance of transceiver(s) 155 to electromagnetically isolate a particular antenna.

In one or more embodiments, controller 110, via communication subsystem 104, performs multiple types of over-the-air (OTA) communication with network nodes 164 of external communication system 148. Particular network nodes 164 can be part of communication networks 165 of public land mobile networks (PLMNs) that provide connections to plain old telephone systems (POTS) 166 for voice calls and wide area networks (WANs) 167 for data sessions. WANs 167 can include Internet and other data networks to communication device 100 and second communication device(s) 144a/144b. The particular network nodes 164 can be cellular “cells”, base nodes, or base stations 168 that support cellular OTA communication using RAT as part of a radio access network (RAN). Unlike earlier generations of cellular services, where voice and data were handled using different RATs, both are now integrated with voice being considered one kind of data communication. Conventionally, broadband, packet-based transmission of text, digitized voice, video, and multimedia communication are provided using Fourth generation (4G) RAT of evolved UTMS radio access (E-UTRA), referred to a Long Term Evolved (LTE), although some cellular data service is still being provided by third generation (3G) Universal Mobile Telecommunications Service (UMTS). A fifth generation (5G) RAT, referred to as fifth generation new radio (5G NR), is being deployed to at least augment capabilities of 4G LTE with a yet higher capability of data transfer. Development continues for what will be six generation (6G) RATs and more advanced RATs. With wireless frequency spectrum seemingly ever expanding, additional antennas (151a-151m, 152a-152n) are incorporated to support newer radio access technologies (RATs) and multi band operation. Dual low band (2L) or quad low band (4L) multiple input multiple output (MIMO) operation dictates multiple antennas communicate on multiple bands simultaneously.

In one or more embodiments, network nodes 164 can be access node(s) 169 that support wireless OTA communication. Communication subsystem 104 can receive OTA communication from location services such as provided by global positioning system (GPS) satellites 170. Communication subsystem 104 communicates via OTA communication channel(s) 172a with base stations 168. Communication subsystem 104 communicates via wireless communication channel(s) 172b with access node(s) 169. In one or more particular embodiments, access node(s) 169 supports communication using one or more IEEE 802.11 wireless local area network (WLAN) protocols. Wi-Fi™ is a family of wireless network protocols, based on the IEEE 802.11 family of standards, which are commonly used between user devices and network devices that provide Internet access. In one or more particular embodiments, communication subsystem 104 communicates with one or more locally networked devices 173 via wired or wireless link 172c provided by access node(s) 169. Communication subsystem 104 receives GPS signal(s) 172d broadcast by GPS satellites 170 to obtain geospatial location information.

In one or more embodiments, controller 110, via communication subsystem 104, performs multiple types of OTA communication with local communication system 174. In one or more embodiments, local communication system 174 includes wireless headset 175 and smart watch 176 that are coupled to communication device 100 to form a personal access network (PAN). Communication subsystem 104 communicates via low power wireless communication channel(s) 172e with wireless headset 175. Communication subsystem 104 communicates via second low power wireless communication channel(s) 172f, such as Bluetooth, with smart watch 176 such as used by person 123a. In one or more particular embodiments, communication subsystem 104 communicates with second communication device(s) 144c via wireless link 172g to form an ad hoc network.

Data storage subsystem 106 of communication device 100 includes data storage device(s) 179. Controller 110 is communicatively connected, via system interlink 180, to data storage device(s) 179. Data storage subsystem 106 provides applications, program code, and stored data on nonvolatile storage that is accessible by controller 110. For example, data storage subsystem 106 can provide a selection of applications and computer data such as BPM application 112, video conference application 113 and other application(s) 114 that use communication services. These applications can be loaded into device memory 102 for execution by controller 110. In one or more embodiments, data storage device(s) 179 can include hard disk drives (HDDs), optical disk drives, and/or solid-state drives (SSDs), etc. Data storage subsystem 106 of communication device 100 can include removable storage device(s) (RSD(s)) 181, which is received in RSD interface 182. Controller 110 is communicatively connected to RSD 181, via system interlink 180 and RSD interface 182. In one or more embodiments, RSD 181 is a non-transitory computer program product or computer readable storage device. Controller 110 can access RSD 181 or data storage device(s) 179 to provision communication device 100 with program code, such as code for BPM application 112, video conference application 113, and other application(s) 114.

In one or more embodiments, I/O subsystem 108 includes network interface controller (NIC or “network interface”) 185 with a network connection (NC) 186. Network cable 187 connects NC 186 to wired area network 188. NIC 185 can be referred to as a “network interface” that can support one or more network communication protocols. Wired area network 188 can be a local area network (LAN), a campus area network (CAN), a metropolitan area network (MAN), or a wide area network (WAN). For example, NC 186 can be an Ethernet connection. Second communication devices 144d is communicatively coupled to wired area network 188.

Controller 110 manages, and in some instances directly controls, the various functions and/or operations of communication device 100. These functions and/or operations include, but are not limited to including, application data processing, communication with second communication devices, navigation tasks, image processing, and signal processing. In one or more alternate embodiments, communication device 100 may use hardware component equivalents for application data processing and signal processing. For example, communication device 100 may use special purpose hardware, dedicated processors, general purpose computers, microprocessor-based computers, micro-controllers, optical computers, analog computers, dedicated processors and/or dedicated hard-wired logic.

Controller 110 includes processor subsystem 118, which includes one or more central processing units (CPUs), depicted as data processor 189. Processor subsystem 118 can include one or more digital signal processors 190 that are integrated with data processor 189. Processor subsystem 118 can include other processors that are communicatively coupled to data processor 189, such as baseband processor 163 of communication module 154. In one or embodiments that are not depicted, controller 110 can further include distributed processing and control components that are external to housing 139 or grouped with other components, such as I/O subsystem 108. Data processor 189 is communicatively coupled, via system interlink 180, to device memory 102. In one or more embodiments, controller 110 of communication device 100 is communicatively coupled via system interlink 180 to communication subsystem 104, data storage subsystem 106, and I/O subsystem 108.

System interlink 180 represents internal components that facilitate internal communication by way of one or more shared or dedicated internal communication links, such as internal serial or parallel buses. As utilized herein, the term “communicatively coupled” means that information signals are transmissible through various interconnections, including wired and/or wireless links, between the components. The interconnections between the components can be direct interconnections that include conductive transmission media or may be indirect interconnections that include one or more intermediate electrical components. Although certain direct interconnections (system interlink 180) are illustrated in FIG. 1, it is to be understood that more, fewer, or different interconnections may be present in other embodiments.

According to one aspect, communication device 100 can function as a content-discerning “transmitting device” that operates as a host or presenter, providing one of multiple image streams 193a-193d to communicate to one or more second communication device(s) 144a-144b that function as “receiving device(s)” for remote participants. The multiple image streams 193a-193d can originate from different image capturing devices. In an example, image capturing devices 130a-130b of communication device 100 can provide respective ones of multiple image streams 193a-193b. In another example, at least one of second communication devices 144c-144d is available locally to communication device 100 to provide a respective one or more of image streams 193c-193d to controller 110. Communication device 144d includes image capturing device (ICD) 130d. In an additional example, peripheral device 140 has image capturing device 130e that provides image stream 193e to controller 110. Controller 110 configures communication device 100 and the communicatively connected one or more second communication devices 144c-144e and peripheral device 140 to perform functionality described herein by visually identifying positioning and movement by person 123 captured in one image stream 193a-193e that may be local or remote to communication device 100 According to one embodiment, controller 110 executes BPM application 112 and video conference application 113, stored in a computer readable medium such as device memory 102, to visually detect compliance with policies by person 123 using communication device 100 or one of second communication device(s) 144a-144d.

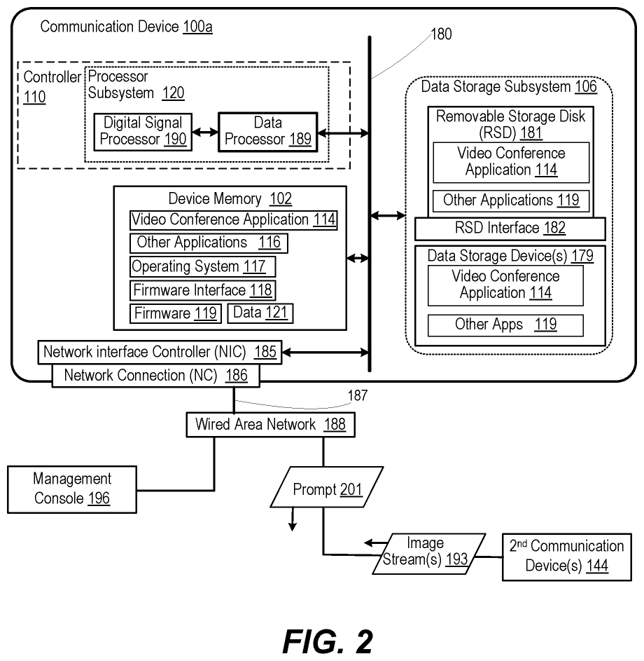

FIG. 2 depicts a functional block diagram of a communication environment including a network communication device 100a having network server components that facilitates a video communication session and that automatically manages healthy participation by a participant who is using a second communication device, and within which the features of the present disclosure are advantageously implemented. In one or more embodiments, communication device 100a can have similar or identical components to communication device 100 with certain components unused or removed to enable communication device 100a to function as a network server. In one or more embodiment, communication device 100a is purpose assembled as a network server having controller 110 that manages device memory 102, data storage subsystem 106, and NIC 185. Management console 196 can periodically communicatively connect to communication device 100a via wired area network 188 to configure program code such as BPM application 112 and video conference application 113 and data 119. Controller 110 executes video conference application 113 to configure communication device 100a to facilitate one or more communication sessions for second communication device(s) 144. In an example, second communication device(s) 144 are not configured to monitor healthy/ergonomic positioning and movement of a person using each second communication device 144. In one or more embodiments, communication device 100a provides the functionality for prompting healthy body positioning and movement during computer usage. Communication device 100a receives image stream(s) 193 from second communication device(s) 144 and identifies positioning and/or body part movement by corresponding person 123 incorporated in each image stream 193. Communication device 100a identifies particular health recommendations applicable to person 123 and communicates or transmits prompt 201 to the corresponding second communication device(s) 144 with the health recommendations.

Aspects of the present disclosure can promote healthy body positioning and movement for situations where a person is standing or sitting in a particular location for an extended period of time. Having at least one output device and at least one image capturing device near the person provides an opportunity for monitoring the person for body position and movement and then presenting customized alerts. In an example, the person is using the communication device as a computer workstation providing one or more applications. In another example, the person may be using a communication device for playing back media content such as a recorded classroom lecture. In an additional example, the person may be using a communication device for a live communication session as an active participant, sharing at least an audio stream with second communication devices. In a further example, the person may be using a communication device for a live communication session as a passive participant who is not called upon to respond. The communication device may not communicate an audio stream or may not communicate a video stream. When the image stream is communicated to another “receiving” communication device, that receiving communication device can provide the functionality of monitoring body positioning and movement of the person. In both instances when the image stream is communicated and when the image stream is not communicated, the local communication device may provide the functionality of monitoring positioning and movement of the person. A video communication session is an example of a situation in which a person may remain in a particular position for an extended period of time but is not the only such situation. The host or presenter of the video communication session may be a teacher, a lecturer, a trainer, a supervisor, an entertainer, a demonstrator, a religious figure, or other functionary. More than one person may take turns presenting visual and/or aural content to the communication session. Media content that is pre-recorded may constitute the entirety or a portion of the visual and/or aural content. Aspects of the present disclosure further apply to haptic or tactile content.

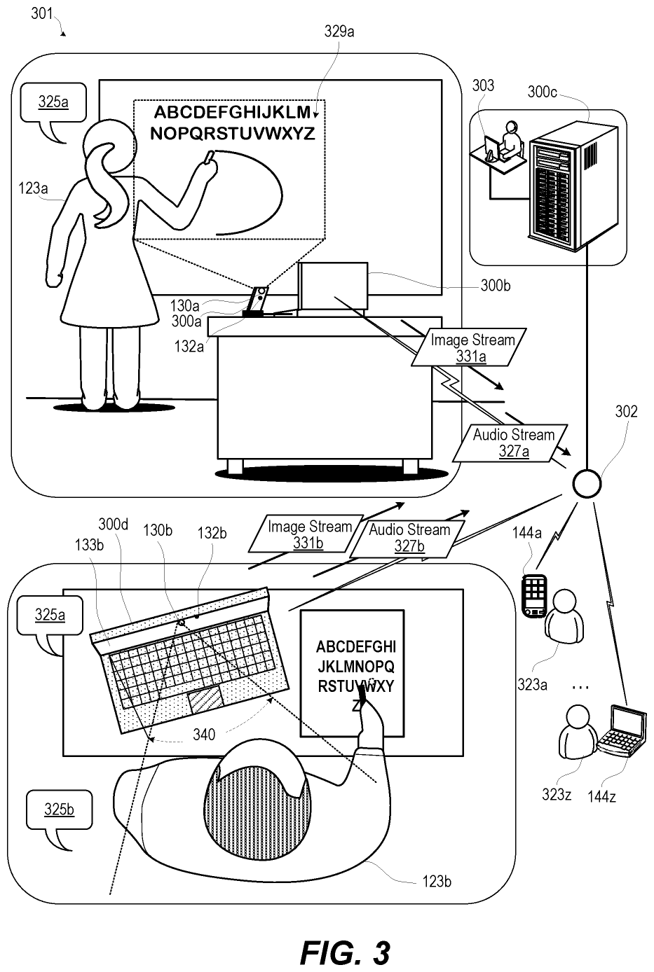

FIG. 3 depicts communication environment 301, which manages monitoring of body positioning and movement of participants during a video communication session conducted across network 302 and provides prompting of health recommendations during the video communication session. For clarity, network 302 is depicted as a single node but may be a combination of wired and wireless connections as described above with regard to FIG. 1. Communication environment 301 includes host/teacher communication devices 300a-300b used by first person 123a that may have similar or identical components and functionality as described for communication device 100 (FIG. 1). Communication device 300a is depicted as a smartphone. Communication device 300b is depicted as a laptop. Network communication device 300c is generally autonomous but can be controlled by management console 303 on occasion to make configuration changes. Network communication device 300c may have similar identical components and functionality of communication device 100a (FIG. 2). Participant communication device 300d is operated/used by second person 123b. In one or more embodiments, additional second communication device(s) 144a-144z are connected via network 302 to the video communication session and operated/used by corresponding additional participants 323a-323z. First person 123a is creating speech 325a that is captured by microphone 132a of host/teacher communication device 300a and communicated as audio stream 327a to the video communication session. First person 123a is also creating written content 329a that is captured by image capturing device 130a of host/teacher communication device 300a and communicated as image stream 331a to the video communication session. Participant communication device 300d presents audio stream 327a and image stream 331a for second person 123b. Microphone 132b of participant communication device 300d captures speech 325b by second person 123b as second audio stream 327b and may communicate second audio stream 327b to the video communication session if microphone function is not muted at participant communication device 300d. Image capturing device 130b of participant communication device 300d has a field of view (FOV) 340 that captures second image stream 331b, which incorporates second person 123b. Participant communication device 300d may communicate second image stream 331b to the video communication session if image capturing device 130b is not disabled at participant communication device 300d. Notably, even if second audio stream 327b and second image stream 331b are transmitted to and received by host/teacher communication devices 300a-300b, host/teacher communication devices 300a-300b may not present second audio stream 327b and second image stream 331b to first person 123a or to other second persons participating in the video communication session. In addition, first person 123a may be occupied with presenting instructional content and be unable to monitor positioning of and/or movement by second person 123b. Prompting for healthy positioning and movement may be performed in conjunction with monitoring other visually detected compliance factors such as attendance, attentiveness, proper attire, etc. Alternatively, prompting for healthy positioning and movement may be performed in situations in which attendance, attentiveness, proper attire, etc., are not tracked. Ergonomic compliance may be individually scored and reported as part of automated participant performance monitoring. Automated affirmations or cautions may be presented to participants to encourage proper ergonomic behaviors. In an example, messages are presented: (i) “You have been identified as a participant with best seating posture, well done!” (ii) “You have been blinking better that 40% of the participants, perhaps you should blink more often!” (iii) “There is an external noise detected which seem to be not related to this call and might disturb other participants, consider muting yourself!” In one or more embodiments, the messages are private to the applicable participant. In one or more embodiments, the ergonomic monitoring is furtherance of a video communication session. Non-compliance is flagged and reported to a host participant such as a trainer, teacher, or supervisor to support discussions during the session or later for improved behavior. In one or more embodiments, the reporting does not occur until after the participant has failed to respond to a private prompt.

Second person 123b may tend to be sedentary during the video communication sessions or to stare at participant communication device 300d for extended periods. According to different embodiments, one or more of host/teacher communication devices 300a-300b, network communication device 300c, and participant communication device 300d can perform the functionality of monitoring and prompting health recommendations related to positioning and/or movement of second person 123b. In an example, to reduce transmission and computational overhead for host/teacher communication devices 300a-300b and network communication device 300c, participant communication device 300d can be configured to monitor image stream 331b from image capturing device 130b and report health recommendation alerts to host/teacher communication devices 300a-300b and/or network communication device 300c. In one or more embodiments, one or more of host/teacher communication devices 300a-300b and network communication device 300c receive second image stream 331b and manage transmission of health prompts to participant communication device 300d, enabling participant communication device 300d to present health prompts even if participant communication device 300d is not specifically configured or equipped to perform the functionality of the present disclosure.

In one or more embodiments,

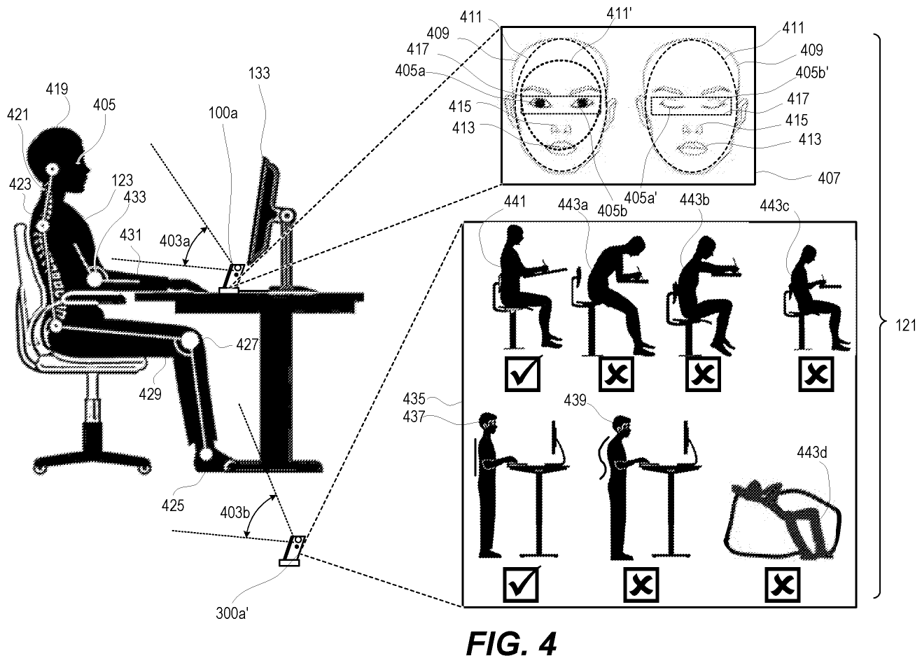

FIG. 4 depicts communication device 300a positioned to have FOV 403a for visually identifying position and/or movement by person 123. In one embodiment, communication device 300a is positioned to detect position of eyes 405 of face 409 of person 123. Communication device 300a is equipped with face visual library 407, which can be a subset of BPM visual library 121. Face visual library 407 provides facial recognition template or recognition rules for identifying face shape 411 that matches or is substantially similar to face 409. In an example, face visual library 407 can provide other face shapes 411′ that do not match face 409 but would match other faces. In one or more embodiments, communication device 300a determines distance to face 409 and defines face shape 411 as being within a pre-defined range of face sizes. Facial features, such as mouth 413, nose 415, and left and right eyes 405a, 405b, may be identified to be within a certain portion of face shape 411. As shown, left and right eyes 405a-405b are within eye box area 417 of face shape 411 and left and right eyes 405a-405b are open. Controller 110 (FIG. 1) of communication device 300a detects when left and right eyes 405a-405b become closed eyes 405a′-405b′, such as when the person is blinking or when left and right eyes 405a-405b are closed for an extended period.

In another example, communication device 100b is positioned to detect posture of person 123 to be in one or more sitting and standing positions. In an example, FOV 403b of communication device 100b can detect an ergonomic (straight) posture of head 419, neck 421 and back 423. In another example, communication device 300a′ can detect one or more of: (i) feet 425 being flat on the floor or on a foot support; (ii) knees 427 bent between 90-120°; (iii) thighs 429 horizontal; (iv) arms 431 supported; (v) elbows 433 bent between 90-120°; (vi) display device 133 positioned about 18-24 inches and level with eyes 405. In one or more embodiments, communication device 100b visually compares the current posture of person 123 to posture visual library 435, which can be a subset of BPM visual library 121. In an example, posture visual library 435 includes: (i) ergonomically correct standing posture image(s) 437; (ii) ergonomically incorrect standing posture image(s) 439; (iii) ergonomically correct sitting posture image(s) 441; and (iv) ergonomically incorrect sitting posture images 443a-443d.

In one or more embodiments, one or more communication devices 100a-100b is/are positioned to monitor position and movement of person 123 but is/are not facilitating an activity for person 123. In an example, person 123 is watching or listening to media content, is staring out of a window, is reading a book, or engaged in any other activity that tends to be sedentary and/or that enables bad posture. One or more communication devices 100a-100b may visually monitor person 123 and present, or cause to be presented, an alert to comply with a health recommendation. In an example, communication device 100b may be placed in an assistive living situation wherein one or more person 123 may remain seated for an extended period. An alert may prompt a particular person 123 to move when recommended or alert a care giver to prompt the particular person 123. The amount of time for each person 123 may be customized. In an example, person 123 may have a circulatory situation in which blood clots in the legs may occur in a shorter time when not taking an anticoagulant drug. One or more communication devices 100a-100b may access settings specific to each identified person 123 within an image stream.

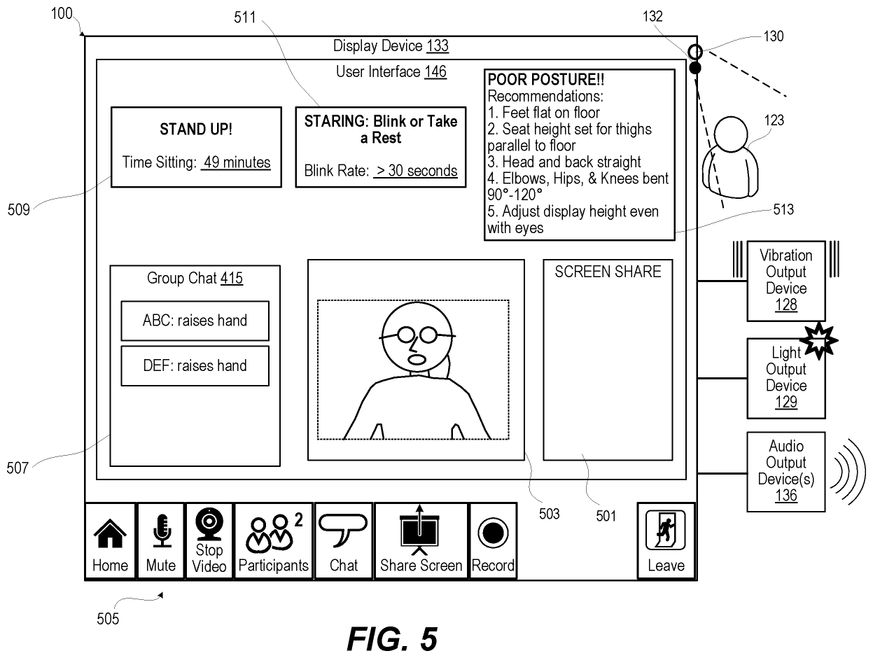

FIG. 5 depicts communication device 100 having user interface 146 that presents prompts related to health recommendations for positioning and/or movement of person 123 who uses communication device 100. Visual content such as screen share 501 and image stream 503 are presented on display device 133 of communication device 100. In an example, user interface 146 facilitates participation in a one-way or two-way video communication session by having video conference controls 505 such as: (i) “home” control; (ii) mute/unmute toggle control; (iii) stop/start video toggle control; (iv) view mode control; (v) chat window toggle control; (vi) share/unshare screen toggle control; (vii) start/stop record toggle control; and (viii) leave session control. User interface 146 can include chat interface 507. Image capturing device 130 of communication device 100 monitors position/movement of person 123. Communication device 100 presents one or more different alerts 509-513 to prompt person 123 to comply with health recommendations. In an example, alert 509 indicates how long person 123 has remained sitting and that person 123 should stand to ensure proper blood circulation in the legs. In another example, alert 511 indicates how long person 123 has not blinked and should blink or take a rest. In an additional example, alert 513 indicates that person 123 is not standing or sitting in an ergonomically correct position. To enhance the likelihood of the alert being noticed by person 123, vibration output device 128 can vibrate. Similarly, light output device 129 can illuminate, such as with a blinking or colored light output, to gain the attention of person 123. Alternatively, or in addition, audio output device(s) 136 can produce a tone or instructional audio outputs to prompt person 123.

FIG. 5 depicts user interface 146, which presents alerts on display device 133 of communication device 100. In an example, user interface 146 provides control and user settings for a video communication session. First person 123 may operate communication device 100 by being positioned within detection range of image capturing device 130 and microphone 132 to produce an audiovisual stream that is communicated to second communication device(s) 144a-144b (FIG. 1). User interface 146 provides general video communication session controls 505 such as “home” for settings, audio mute/unmute toggle, stop/start video toggle, view mode toggle, chat window selection, share screen toggle, record start/stop control, and video communication session join/leave toggle.

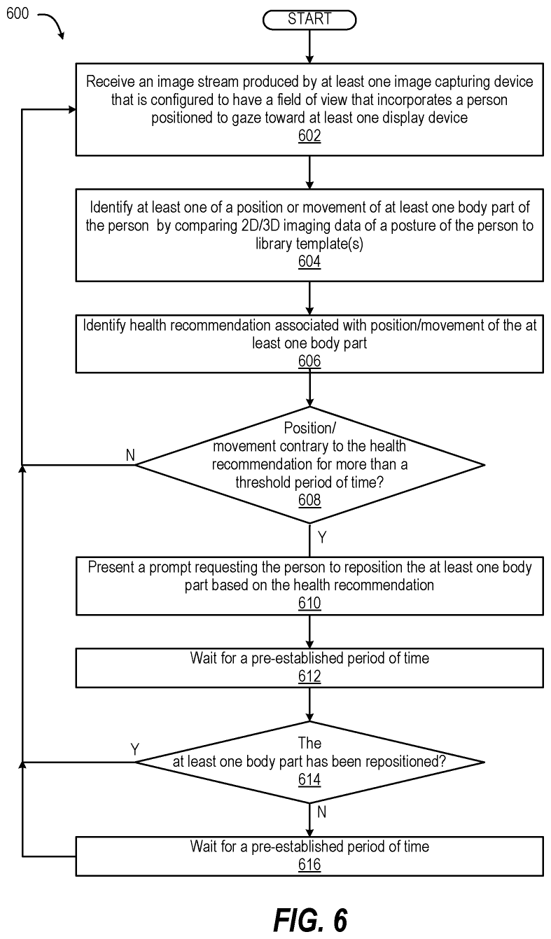

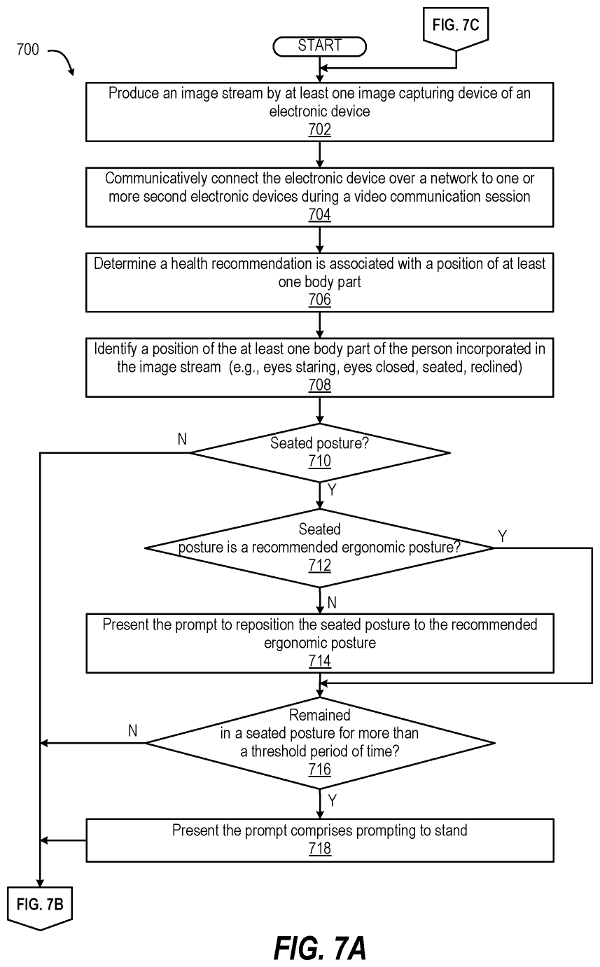

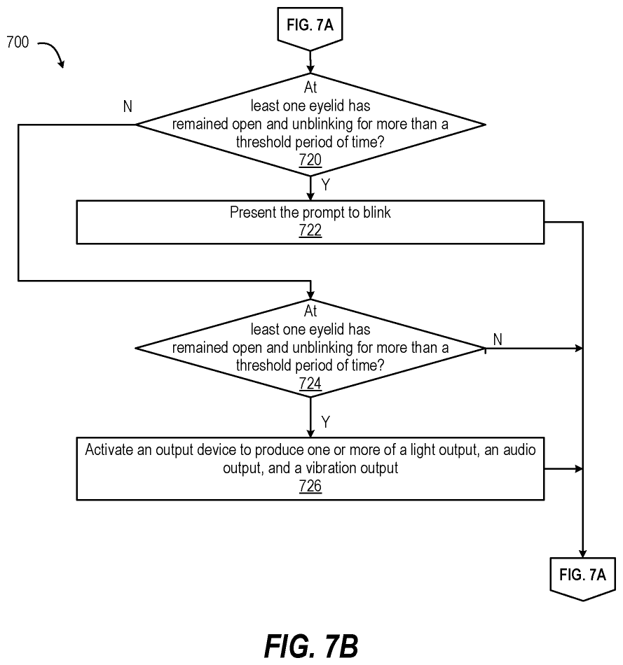

With reference now to the flow charts, there are presented method 600 (FIG. 6) and method 700 (FIGS. 7A-7B). Method 600, depicted in FIG. 6, describe examples of functionality performed by one or more of host communication devices 300a-300b used by person 123a, network communication device 300c, or communication device 300d which is used by person 123b (FIG. 3). The functionality includes prompting healthy positioning and movement of person 123b (FIG. 3). Method 700, depicted in FIGS. 7A-7B (collectively FIG. 7), provides for monitoring more than one health recommendation for prompting healthy positioning and movement of person 123b who uses communication device 300d (FIG. 3). The descriptions of methods 600 and 700 are provided with general reference to the specific components illustrated within the preceding FIGS. 1-5, and specific components referenced in methods 600 and 700 may be identical or similar to components of the same name used in describing preceding FIGS. 1-5.

With reference to FIG. 6, method 600 includes receiving an image stream produced by at least one image capturing device, which is configured to have a field of view that incorporates a person positioned to gaze toward at least one display device (block 602). In one or more embodiments, aspects of the present disclosure assist the person who may be doing an activity that does not encourage or require gazing toward a display device. Method 600 includes identifying at least one of a position or movement of at least one body part of the person incorporated in the image stream by comparing one or more of two-dimensional (2D) and three-dimensional (3D) imaging data of a posture or eye movement of the person to one or more library templates (block 604). Method 600 includes determining at least one health recommendation associated with the position and/or movement of the at least one body part (e.g., unblinking open eyes, closed eyes, extended sitting, sitting in a non-ergonomic slouching posture, standing in a non-ergonomic posture) (block 606).

Method 600 includes determining whether the position and/or movement of the at least one body part is not in accordance with one or more pre-established health recommendations for more than a threshold period of time (decision block 608). In one or more embodiments, a different threshold period of time is applicable to each of at least one body part, body position, and/or movement. It is appreciated that method 600 may monitor concurrently for more than one health recommendation. In one or more embodiments, the health recommendation is further customized for a particular demographic category or pre-determined health history of the person.

In response to the determining that the position and/or movement of the at least one body part is not out of compliance with the health recommendation for more than the threshold period of time, method 600 returns block 602. In response to determining that the position and/or movement of the at least one body part is not in accordance with the health recommendation for more than the threshold period of time, method 600 includes presenting a prompt requesting the person to reposition the at least one body part based on the health recommendation (block 610). The prompt can be presented on one or more of at least one display device and at least one audio output device. In one or more embodiments, method 600 includes presenting a second prompt on one or more of a light output device (e.g., blinking or colored light), an audio output device (e.g., alert tone), and a vibration output device to get the attention of the person who may not be paying attention to the at least one display device.

Method 600 includes waiting for a pre-established period of time (block 612). In an example, the pre-established period of time can be different for each different healthcare recommendation. For example, a recommendation to stand can be less time critical, allowing a longer pre-established period of time to elapse, such as 5-15 minutes, allowing the person to wait for an appropriate moment to stand. In another example, an inability to wake the person may indicate a serious health incident that requires a response with 15-60 seconds. With this example, failure of the person to respond to the prompt may indicate a more serious health condition that requires intervention by others. The health condition can be a known condition, and the method thus implements this periodic check on the participant's alertness/attentiveness, in part based on the health information known about the particular participant. Method 600 includes identifying whether the at least one body part has been repositioned, in response to and/or based on the prompt (decision block 614). In an example, blinking after being prompted to blink is an example of repositioning. In another example, standing after being prompted to stand is an example of repositioning. In a further example, opening the eyes after being prompted to open the eyes is an example of repositioning. In an additional example, straightening the head, neck, and back into an ergonomic posture after being prompted to straighten the posture is an example of repositioning. In yet another example, standing up after being prompted to change a poor ergonomic sitting posture is an example of repositioning. Adjusting seat heights for proper alignment of legs, knees and feet when prompted to correct leg position to a proper ergonomic position is an example of repositioning. In response to identifying that the at least one body part has been repositioned, method 600 returns to block 602. In response to identifying that the at least one body part has not been repositioned, method 600 includes communicating an alert over a network to a host electronic device (block 616). Then method 600 returns to block 602.

FIGS. 7A-7B (FIG. 7) presents a flow diagram of method 700, which includes responding to particular examples of healthcare recommendations related to positioning and moving body parts of a person. With reference to FIG. 7A, method 700 includes producing an image stream by at least one image capturing device of an electronic device (block 702). The at least one image capturing device is configured to have a field of view that incorporates a person positioned to gaze toward at least one display device. Method 700 includes communicatively connecting the electronic device over a network to one or more second electronic devices during a video communication session (block 704). Method 700 includes determining a health recommendation is associated with a position of at least one body part (block 706). Method 700 includes identifying a position of the at least one body part of the person incorporated in the image stream (e.g., eyes staring, eyes closed, seated with a non-ergonomic position, or standing with a non-ergonomic posture) (block 708). Method 700 includes determining whether the at least one body part is identified with the person being in a seated posture (decision block 710). In response to determining that the at least one body part is not identified with the person being in a seated posture, method 700 proceeds to decision block 720 (FIG. 7B). In response to determining that the at least one body part indicates that the person is in a seated posture, method 700 includes determining whether the seated posture is a recommended ergonomic posture (decision block 712). In response to determining that the seated posture is a recommended ergonomic posture, method 700 proceeds to decision block 716. In response to determining that the seated posture is not a recommended ergonomic posture, method 700 includes presenting a prompt for the person to reposition the seated posture to the recommended ergonomic posture (block 714). In response to determining that the seated posture is a recommended ergonomic posture in decision block 712 or after block 714, method 700 includes determining whether the person has remained in a seated posture for more than a threshold period of time (decision block 716). In response to determining that the person has not remained in a seated posture for more than a threshold period of time, method 700 proceeds to decision block 720 (FIG. 7B). In response to determining that the person has remained in a seated posture for more than a threshold period of time, method 700 includes presenting a prompt that includes prompting the person to stand. Then method 700 proceeds to decision block 720 (FIG. 7B). Method 700 proceeds to decision block 720 after the “No” exit from either decision block 710 and 716 or after block 718.

With reference to FIG. 7B, method 700 includes determining whether at least one eyelid has remained open and unblinking for more than a threshold period of time related to the minimum rate of blinking (decision block 720). In response to determining that the at least one eyelid has remained open and unblinking for more than a threshold period of time related to the minimum rate of blinking, method 700 includes presenting the prompt requesting the person to blink (block 722). Then method 700 returns to block 702 (FIG. 7A).

In response to determining that the at least one eyelid has not remained open and unblinking for more than a threshold period of time related to the minimum rate of blinking, method 700 includes determining whether the at least one eyelid has remained closed for more than a second period of time (decision block 724). For clarity, decision block 724 may make determinations on being able to monitor one eye based on positioning of an image capturing device to one lateral side of the person. Additional functionality may be incorporated to detect that a person has a bandaged or covered eye that is not a closed eye. The functionality can include detecting that the person continues to gaze with one eye while the other eye is closed, such as to accommodate an irritated eye. The functionality may include determining that the particular person is visually impaired and that this health recommendation is not applicable to the person. In response to determining that the at least one eyelid has remained closed for more than the second period of time, method 700 includes activating an output device to produce one or more of a light output, an audio output, and a vibration output (block 726). In response to determining that the at least one eyelid has not remained closed for more than the second period of time, method 700 returns to block 702 (FIG. 7A).

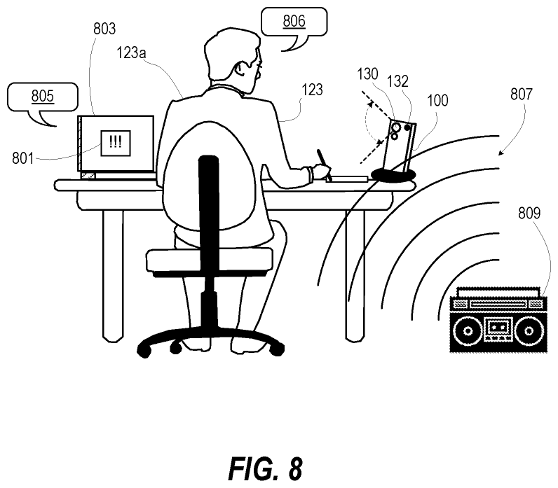

FIG. 8 depicts communication device 100 providing background noise alerts 801-802, respectively, on external display device 803 and on communication device 100 to person 123. Excessive background noise may violate a health recommendation for protecting hearing of person 123. Reducing excessive background noise can further avoid a distraction to other persons who may receive an audio signal from communication device 100 and/or avoid person 123 having a degraded learning (or communication) experience. In one or more embodiments, communication device 100 dynamically and periodically evaluates background noise by determining what sounds 805-806 are intentionally produced respectively by communication device 100 and person 123 and what sounds 807 are produced by an external source such as audio output player 809. In one or more embodiments, communication device 100 can determine a source of each sound 805-807 by at least one: (i) identifying an originating location of the sound; (ii) recognizing the sound; and (iii) comparing an audio signal that is communicated to an output device such as external display device 803 to an audio input sensed by microphone 132. Communication device 100 can compare the current background noise level to one or more thresholds such as: (i) a first threshold below which it is allowable to share an audio stream to a video communication session; (ii) a second threshold that should not be exceeded for more than 30 seconds to avoid a personal distraction to person 123; and (iii) a third threshold that should not be exceeded for more than 10 minutes to avoid temporary hearing loss.

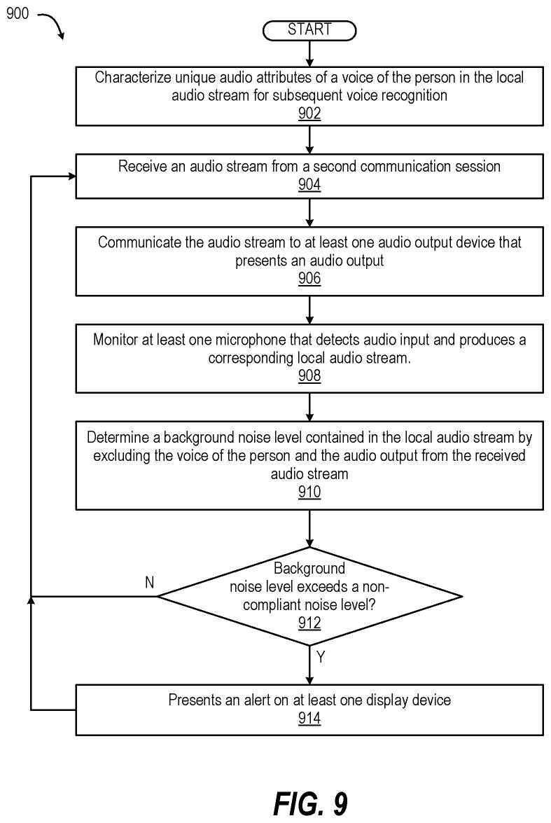

FIG. 9 depicts a flow diagram of method 900 performed by communication device 100 for prompting avoidance of an excessively noisy environment. Method 900 can be performed independently or in conjunction with monitoring of positioning and movement of person 123 (FIG. 8). The descriptions of method 900 is provided with general reference to the specific components illustrated within the preceding FIGS. 1-6, 7A-7B, and 8, and specific components referenced in method 900 may be identical or similar to components of the same name used in describing preceding FIGS. 1-6, 7A-7B, and 8. Method 900 includes characterizing unique audio attributes of a voice of the person in the local audio stream for subsequent voice recognition (block 902). Method 900 includes receiving an audio stream from a second communication session (block 904). Method 900 includes communicating the audio stream to at least one audio output device that presents an audio output (block 906). Method 900 includes monitoring at least one microphone that detects audio input and produces a corresponding local audio stream (block 908). Method 900 includes determining a background noise level contained in the local audio stream by excluding the voice of the person and the audio output from the received audio stream (block 910). Method 900 includes determining whether the background noise level exceeds a non-compliant noise level (decision block 912). In response to the background noise level not exceeding the non-compliant noise level, method 900 returns to block 904. In response to the background noise level not exceeding the non-compliant noise level, method 900 includes presenting an alert on at least one display device (block 914). Then method 900 returns to block 904.

In one or more embodiments, method 900 includes receiving an image stream produced by at least one image capturing device that is configured to have a field of view that incorporates a person positioned to gaze toward at least one display device. Method 900 includes identifying at least one of a position or movement of at least one body part of the person incorporated in the image stream. Method 900 includes determining whether a health recommendation is associated with the position and/or movement of the at least one body part. Method 900 includes presenting a prompt at the at least one display device requesting the person to reposition the at least one body part based on the health recommendation being associated with the position and/or movement of the at least one body part.

In one or more embodiments, method 900 further includes identifying the position of the at least one body part by comparing one of two-dimensional and three-dimensional imaging data of a posture of the person to one or more library templates that are each identified as one of ergonomically correct and ergonomically incorrect. Method 900 includes determining whether the health recommendation is associated with the position by determining whether the posture is a recommended ergonomic posture. Method 900 includes presenting the prompt to request the person reposition at least the one body part in order to have a posture that aligns with the recommended ergonomic posture.

In one or more embodiments, method 900 further includes the at least one body part is at least one eyelid. The health recommendation is a minimum rate of blinking of an eyelid. Method 900 includes determining whether the at least one eyelid has remained open or closed and unblinking for more than a blinking threshold period of time corresponding to the minimum rate of blinking. Method 900 includes presenting the prompt notifying the person at least one of: (i) to blink; and (ii) to take a rest break.

In one or more embodiments, method 900 further includes identifying the position of the at least one body part by detecting a seated posture. Method 900 includes determining whether the health recommendation is associated with the position by determining whether the person has remained in a seated position for more than a sitting threshold period of time. Method 900 includes presenting the prompt notifying the person to stand based on determining that the person has remained in a seated position for more than the sitting threshold period of time.

In one or more embodiments, method 900 further includes presenting the prompt by: (i) triggering one or more of the at least one output device to generate a corresponding output from among the light output, the audio output, and the vibration output; and (ii) presenting one or more of a notification with specific information about a change that is required via one or more of the at least one display device and the audio output device.

In one or more embodiments, method 900 further includes: (i) identifying whether the at least one body part has been repositioned, in response to the prompt, within a pre-established response period of time; and (ii) communicating an alert over a network to a host electronic device in response to determining that the at least one body part has not been repositioned within the response period of time.

In one or more embodiments, method 900 further includes monitoring at least one eyelid of the person as the at least one body part. Method 900 includes determining whether the at least one eyelid has remained closed for more than a threshold period of time. Method 900 includes activating an output device that produces one or more of a light output, an audio output, and a vibration output in an attempt to wake the person. In one or more particular embodiments, method 900 further includes: (i) determining whether the person has opened the at least one eyelid within a response threshold period of time following the activation of the output device; and (ii) communicating an alert over the network to a host electronic device in response to determining that the person has not opened the at least one eyelid within the response period of time.

In one or more embodiments, method 900 further includes monitoring at least one microphone that detects audio input and produces a corresponding local audio stream. Method 900 includes characterizing unique audio attributes of a voice of the person in the local audio stream for subsequent voice recognition. During the video communication session, method 900 includes: (i) receiving, via the at least one network interface and the network, a received audio stream from the video communication session; (ii) presenting the received audio stream at the at least one audio output device as an audio output; (iii) determining a background noise level contained in the local audio stream by excluding the voice of the person and the audio output from the received audio stream; and (iv) presenting an alert on the at least one display device in response to the background noise level exceeding a noise threshold level related to the health recommendation.

Aspects of the present innovation are described above with reference to flowchart illustrations and/or block diagrams of methods, apparatus (systems) and computer program products according to embodiments of the innovation. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

As will be appreciated by one skilled in the art, embodiments of the present innovation may be embodied as a system, device, and/or method. Accordingly, embodiments of the present innovation may take the form of an entirely hardware embodiment or an embodiment combining software and hardware embodiments that may all generally be referred to herein as a “circuit,” “module” or “system.”

While the innovation has been described with reference to exemplary embodiments, it will be understood by those skilled in the art that various changes may be made, and equivalents may be substituted for elements thereof without departing from the scope of the innovation. In addition, many modifications may be made to adapt a particular system, device, or component thereof to the teachings of the innovation without departing from the essential scope thereof. Therefore, it is intended that the innovation not be limited to the particular embodiments disclosed for carrying out this innovation, but that the innovation will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the innovation. As used herein, the singular forms “a”, “an” and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present innovation has been presented for purposes of illustration and description but is not intended to be exhaustive or limited to the innovation in the form disclosed. Many modifications and variations will be apparent to those of ordinary skill in the art without departing from the scope and spirit of the innovation. The embodiments were chosen and described in order to best explain the principles of the innovation and the practical application, and to enable others of ordinary skill in the art to understand the innovation for various embodiments with various modifications as are suited to the particular use contemplated.

What is claimed is:

1. An electronic device comprising:
at least one display device;
at least one image capturing device that produces an image stream and is configured to have a field of view that incorporates a person positioned to gaze at the at least one display device; and
a controller communicatively coupled to the at least one display device and the at least one image capturing device, and which:
identifies at least one of a position or movement of at least one body part of the person incorporated in the image stream;
determines whether a health recommendation is associated with the position and/or movement of the at least one body part; and
presents a prompt at the at least one display device requesting the person to reposition the at least one body part based on the health recommendation being associated with the position and/or movement of the at least one body part.

2. The electronic device of claim 1, wherein the controller:
identifies the position of the at least one body part by comparing one of two-dimensional and three-dimensional imaging data of a posture of the person to one or more library templates that are each identified as one of ergonomically correct and ergonomically incorrect;
determines whether the health recommendation is associated with the position by determining whether the posture is a recommended ergonomic posture; and
presents the prompt to request the person reposition at least the one body part in order to have a posture that aligns with the recommended ergonomic posture.

3. The electronic device of claim 1, wherein:
the at least one body part is at least one eyelid;
the health recommendation is a minimum rate of blinking of an eyelid; and
the controller:
determines whether the at least one eyelid has remained open or closed and unblinking for more than a blinking threshold period of time corresponding to the minimum rate of blinking; and
presents the prompt notifying the person at least one of: (i) to blink; and (ii) to take a rest break.

4. The electronic device of claim 1, wherein the controller:
identifies the position of the at least one body part by detecting a seated posture;
determines whether the health recommendation is associated with the position by determining whether the person has remained in a seated position for more than a sitting threshold period of time; and
presents the prompt notifying the person to stand based on determining that the person has remained in a seated position for more than the sitting threshold period of time.

5. The electronic device of claim 1, further comprising at least one output device communicatively coupled to the controller and that produces one or more of a light output, an audio output, and a vibration output, wherein the controller presents the prompt by:
triggering one or more of the at least one output device to generate a corresponding output from among the light output, the audio output, and the vibration output; and
presenting one or more of a notification with specific information about a change that is required via one or more of the at least one display device and the audio output device.

6. The electronic device of claim 1, further comprising at least one network interface communicatively coupled to the controller and that communicatively connects the electronic device over a network to one or more second electronic devices during a video communication session, and the controller:
identifies whether the at least one body part has been repositioned, in response to the prompt, within a pre-established response period of time; and
communicates an alert over the network to a host electronic device in response to determining that the at least one body part has not been repositioned within the response period of time.

7. The electronic device of claim 1, further comprising an output device communicatively coupled to the controller and that produces one or more of a light output, an audio output, and a vibration output, wherein the controller:
monitors at least one eyelid of the person as the at least one body part;
determines whether the at least one eyelid has remained closed for more than a threshold period of time; and
activates the output device in an attempt to wake the person.

8. The electronic device of claim 1, further comprising at least one network interface communicatively coupled to the controller and that communicatively connects the electronic device over a network to one or more second electronic devices during a video communication session; wherein the controller:
determines whether the person has repositioned the at least one body part within a response threshold period of time following presentation of the prompt; and
communicates an alert over the network to a host electronic device in response to determining that the person has not repositioned the at least one body part within the response period of time.

9. The electronic device of claim 1, further comprising:
at least one audio output device; and
at least one microphone that detects audio input and produces a corresponding local audio stream, the at least one audio output device and the at least one microphone communicatively coupled to the controller, wherein the controller:
characterizes unique audio attributes of a voice of the person in the local audio stream for subsequent voice recognition; and
during the video communication session:
receives, via the at least one network interface and the network, a received audio stream from the video communication session;
presents the received audio stream at the at least one audio output device as an audio output;
determines a background noise level contained in the local audio stream by excluding the voice of the person and the audio output from the received audio stream; and
presents an alert on the at least one display device in response to the background noise level exceeding a non-compliant noise level.

10. A method comprising:
receiving an image stream produced by at least one image capturing device that is configured to have a field of view that incorporates a person positioned to gaze toward at least one display device;
identifying at least one of a position or movement of at least one body part of the person incorporated in the image stream;
determining whether a health recommendation is associated with the position and/or movement of the at least one body part; and
presenting a prompt at the at least one display device requesting the person to reposition the at least one body part based on the health recommendation being associated with the position and/or movement of the at least one body part.

11. The method of claim 10, further comprising:
identifying the position of the at least one body part by comparing one of two-dimensional and three-dimensional imaging data of a posture of the person to one or more library templates that are each identified as one of ergonomically correct and ergonomically incorrect;
determining whether the health recommendation is associated with the position by determining whether the posture is a recommended ergonomic posture; and
presenting the prompt to request the person reposition at least the one body part in order to have a posture that aligns with the recommended ergonomic posture.

12. The method of claim 10, wherein:
the at least one body part is at least one eyelid;
the health recommendation is a minimum rate of blinking of an eyelid; and
the method further comprises:
determining whether the at least one eyelid has remained open or closed and unblinking for more than a blinking threshold period of time corresponding to the minimum rate of blinking; and
presenting the prompt notifying the person at least one of: (i) to blink; and (ii) to take a rest break.

13. The method of claim 10, further comprising:
identifying the position of the at least one body part by detecting a seated posture;
determining whether the health recommendation is associated with the position by determining whether the person has remained in a seated position for more than a sitting threshold period of time; and
presenting the prompt notifying the person to stand based on determining that the person has remained in a seated position for more than the sitting threshold period of time.

14. The method of claim 10, wherein presents the prompt by:
triggering one or more of the at least one output device to generate a corresponding output from among the light output, the audio output, and the vibration output; and
presenting one or more of a notification with specific information about a change that is required via one or more of the at least one display device and the audio output device.

15. The method of claim 10, further comprising:
identifying whether the at least one body part has been repositioned, in response to the prompt, within a pre-established response period of time; and
communicating an alert over a network to a host electronic device in response to determining that the at least one body part has not been repositioned within the response period of time.

16. The method of claim 10, further comprising:
monitoring at least one eyelid of the person as the at least one body part;
determining whether the at least one eyelid has remained closed for more than a threshold period of time; and
activating an output device that produces one or more of a light output, an audio output, and a vibration output in an attempt to wake the person.

17. The method of claim 10, further comprising:
communicatively connecting the electronic device over a network to one or more second electronic devices during a video communication session;
determining whether the person has repositioned the at least one body part within a response threshold period of time following presentation of the prompt; and
communicating an alert over the network to a host electronic device in response to determining that the person has not repositioned the at least one body part within the response period of time.

18. The method of claim 10, further comprising:
monitoring at least one microphone that detects audio input and produces a corresponding local audio stream;
characterizing unique audio attributes of a voice of the person in the local audio stream for subsequent voice recognition; and
during the video communication session:
receiving, via the at least one network interface and the network, a received audio stream from the video communication session;
presenting the received audio stream at the at least one audio output device as an audio output;
determining a background noise level contained in the local audio stream by excluding the voice of the person and the audio output from the received audio stream; and
presenting an alert on the at least one display device in response to the background noise level exceeding a noise threshold level related to the health recommendation.

19. A computer program product comprising:
a computer readable storage device; and
program code on the computer readable storage device that when executed by a processor associated with an electronic device, the program code enables the electronic device to provide functionality of:
receiving an image stream produced by at least one image capturing device that is configured to have a field of view that incorporates a person positioned to gaze toward at least one display device;
identifying at least one of a position or movement of at least one body part of the person incorporated in the image stream;
determining whether a health recommendation is associated with the position and/or movement of the at least one body part; and
presenting a prompt at the at least one display device requesting the person to reposition the at least one body part based on the health recommendation being associated with the position and/or movement of the at least one body part.

20. The computer program product of claim 19, wherein the program code enables the electronic device to provide the functionality of:
identifying the position of the at least one body part by comparing one of two-dimensional and three-dimensional imaging data of a posture of the person to one or more library templates that are each identified as one of ergonomically correct and ergonomically incorrect;
determining whether the health recommendation is associated with the position by determining whether the posture is a recommended ergonomic posture; and
presenting the prompt to request the person reposition at least the one body part in order to have a posture that aligns with the recommended ergonomic posture.
